(19) United States
(12) Patent Application Publication
(10) Pub. No.: US 2024/0100034 A1
(43) Pub. Date: Mar. 28, 2024

(54) METHOD FOR ERADICATING HELICOBACTER PYLORI INFECTION IN PATIENTS REGARDLESS OF BODY MASS INDEX

(71) Applicant: RedHill Biopharma Ltd., Tel-Aviv (IL)

(72) Inventors: Reza Fathi, Oradell, NJ (US); Kely Lee Sheldon, Atlanta, GA (US)

(21) Appl. No.: 18/532,876

(22) Filed: Dec. 7, 2023

Related U.S. Application Data
(63) Continuation of application No. 16/869,199, filed on May 7, 2020, now Pat. No. 11,878,011.

(51) Int. Cl. A61K 31/4439; A61K 9/20; A61K 9/28; A61K 31/43; A61K 31/438

(52) U.S. Cl. CPC A61K 31/4439; A61K 9/2072; A61K 9/28; A61K 31/43; A61K 31/438

(57) ABSTRACT

A method of treating patients who are positive for Helicobacter pylori infection comprises determining that a first patient who is positive for Helicobacter pylori infection has a body mass index corresponding to normal weight or overweight; determining that a second patient who is positive for Helicobacter pylori infection has a body mass index corresponding to obese or extremely obese; and administering to both the first patient and the second patient, for 14 days, a rifabutin-based triple therapy consisting essentially of rifabutin, amoxicillin and omeprazole or an equivalent amount of a pharmaceutically acceptable salt thereof, wherein, after treatment is complete, there is no substantial difference in the efficacy of the treatment when the treatment is administered to the first patient or the second patient.

### CROSS-REFERENCE TO RELATED APPLICATIONS

This application is a continuation of U.S. patent application Ser. No. 16/869,199, filed May 7, 2020, which is incorporated by reference in its entireties for all purposes.

### BACKGROUND

Gastric bypass and other weight-loss surgeries — known collectively as bariatric surgery, remain effective therapies for patients with morbid obesity. They have been shown to be safe and adequate in inducing sustainable and important weight loss. Bariatric surgeons often advocate preoperative Helicobacter pylori (H. pylori) testing and eradication because of the increased risk of postoperative ulcers and foregut symptoms in H. pylori-positive patients. Typically, prior to bariatric surgery, a patient is tested to see if they harbor a H. pylori infection. If the test comes back positive, the patient will have to be treated prior to receiving surgery, delaying their surgery by many months. If the patient's initial treatment for eradicating H. pylori infection is not effective, then that patient will have to try a different therapy, even further delaying their bariatric surgery. Recent evidence suggests patient body mass index (BMI) may contribute to the failure of empiric triple H. pylori therapy and concomitant quadruple H. pylori therapy. Physiologic alterations seen in obesity commonly impact the pharmacokinetics (PK) and pharmacodynamics (PD) of antibiotics typically resulting in suboptimal dosing of drugs and requiring dose modification to achieve efficacy.

### SUMMARY OF THE INVENTION

Surprisingly we have found that the treatment effect observed, eradication of H. pylori infection, when a method of the present invention is carried out with a rifabutin-based H. pylori therapy, is substantially equivalent (i.e., no substantial difference) in adults regardless of their BMI category.

In one aspect, a method of treating patients who are positive for Helicobacter pylori infection comprises determining that a first patient who is positive for Helicobacter pylori infection has a body mass index corresponding to normal weight or overweight; determining that a second patient who is positive for Helicobacter pylori infection has a body mass index corresponding to obese or extremely obese; and administering to both the first patient and the second patient, for 14 days, a rifabutin-based triple therapy consisting essentially of rifabutin, amoxicillin and omeprazole or an equivalent amount of a pharmaceutically acceptable salt thereof, wherein, after treatment is complete, there is no substantial difference in the efficacy of the treatment when the treatment is administered to the first patient or the second patient. In an embodiment, the first patient has a BMI ranging from 18.50 kg/m² to 24.99 kg/m² and the second patient has a BMI ranging from 30.0 kg/m² to 34.99 kg/m^{2 .} In an embodiment, the first patient has a BMI ranging from 18.50 kg/m² to 24.99 kg/m² and the second patient has a BMI ranging from 35.0 kg/m² to 39.99 kg/m^{2 .} In an embodiment, the first patient has a BMI ranging from 18.50 kg/m² to 24.99 kg/m² and the second patient has a BMI above 40.00 kg/m². In an embodiment, the first patient has a BMI ranging from 25.00 kg/m² to 29.99 kg/m² and the second patient has a BMI ranging from 35.0 kg/m² to 39.99 kg/m². In an embodiment, the first patient has a BMI 25.00 kg/m² to 29.99 kg/m² and the second patient has a BMI ranging from 30.0 kg/m² to 34.99 kg/m². In an embodiment, the first patient has a BMI 25.00 kg/m² to 29.99 kg/m² and the second patient has a BMI above 40.00 kg/m^{2 .} In an embodiment, the first patient and the second patient are adults. In an embodiment, the rifabutin-based triple therapy comprises a plurality of fixed-dose combination formulations which provide a total daily dose of 150 mg rifabutin, 3000 mg amoxicillin, and 120 mg omeprazole or an equivalent amount of a pharmaceutically acceptable salt thereof. In an embodiment, the administering comprises taking four fixed-dose combination formulations every 8 hours, wherein each dose of four fixed-dose combination formulations includes 50 mg rifabutin, 1,000 mg amoxicillin and 40 mg omeprazole or an equivalent amount of a pharmaceutically acceptable salt thereof. In an embodiment, each fixed-dose combination formulation comprises 12.5 mg rifabutin, 250 mg amoxicillin and 10 mg omeprazole or an equivalent amount of a pharmaceutically acceptable salt thereof. In an embodiment, each fixed- dose combination formulation is a capsule. In an embodiment, each fixed-dose combination formulation comprises a mixture of amoxicillin/rifabutin immediate release minitablets and enteric-coated omeprazole minitablets. In an embodiment, each minitablet is about 2 mm in size. In an embodiment, each fixed-dose combination formulation comprises between 36 and 40 amoxicillin/rifabutin immediate release minitablets and between 32 and 36 enteric-coated omeprazole minitablets. In an embodiment, each fixed-dose combination formulation comprises 38 amoxicillin/rifabutin immediate release minitablets and 34 enteric-coated omeprazole minitablets. In an embodiment, at least 70% by weight of the omeprazole is intestinally released after oral administration. In an embodiment, the amoxicillin is amoxicillin trihydrate. In an embodiment, the omeprazole is omeprazole magnesium. In an embodiment, the method further comprises confirming H. pylori eradication with a negative ¹³C UBT or fecal antigen test performed ≥28 days post-therapy.

In one aspect, a method for the eradication of Helicobacter pylori infection in an adult comprises administering, each day for 14 days, a plurality of fixed-dose combination formulations which provide a total daily dose of 150 mg rifabutin, 3000 mg amoxicillin, and 120 mg omeprazole or an equivalent amount of a pharmaceutically acceptable salt thereof, wherein, after treatment is complete, there is no substantial difference in the efficacy of the treatment when the treatment is administered to an adult having any BMI classification, including a BMI classification of normal or healthy weight, overweight, obese or extremely obese. In an embodiment, the adult receiving the treatment has a BMI classification of normal with a BMI between 18.5 kg/m² and 24.9 kg/m². In an embodiment, the adult receiving the treatment has a BMI classification of overweight with a BMI≥25.0 kg/m². In an embodiment, the adult receiving the treatment has a BMI classification of pre-obese with a BMI between 25.0 kg/m² and 29.9 kg/m^{2 .} In an embodiment, the adult receiving the treatment has a BMI classification as obese with a BMI≥30.0 kg/m². In an embodiment, the administering comprises taking four fixed-dose combination formulations every 8 hours, wherein each dose of four fixed-dose combination formulations includes 50 mg rifabutin, 1,000 mg amoxicillin and 40 mg omeprazole or an equivalent amount of a pharmaceutically acceptable salt thereof. In an embodiment, each fixed-dose combination formulation comprises 12.5 mg rifabutin, 250 mg amoxicillin and 10 mg omeprazole or an equivalent amount of a pharmaceutically acceptable salt thereof. In an embodiment, each fixed-dose combination formulation is a capsule. In an embodiment, each fixed-dose combination formulation comprises a mixture of amoxicillin/rifabutin immediate release minitablets and enteric-coated omeprazole minitablets. In an embodiment, each minitablet is about 2 mm in size. In an embodiment, each fixed-dose combination formulation comprises between 36 and 40 amoxicillin/rifabutin immediate release minitablets and between 32 and 36 enteric-coated omeprazole minitablets. In an embodiment, each fixed-dose combination formulation comprises 38 amoxicillin/rifabutin immediate release minitablets and 34 enteric-coated omeprazole minitablets. In an embodiment, at least 70% by weight of the omeprazole is intestinally released after oral administration. In an embodiment, the amoxicillin is amoxicillin trihydrate. In an embodiment, the omeprazole is omeprazole magnesium. In an embodiment, the method further comprises confirming H. pylori eradication with a negative ¹³C UBT or fecal antigen test performed ≥28 days post-therapy.

In one aspect, a method for the eradication of Helicobacter pylori infection in an adult comprises administering, each day for 14 days, 12 fixed-dose combination capsules, each capsule comprising 12.5 mg rifabutin, 250 mg amoxicillin and 10 mg omeprazole or an equivalent amount of a pharmaceutically acceptable salt thereof which are present in the capsule as approximately 38 amoxicillin/rifabutin immediate release minitablets and approximately 34 enteric-coated omeprazole minitablets, wherein, after treatment is complete, there is no substantial difference in the efficacy of the treatment when the treatment is administered to an adult having any BMI classification, including a BMI classification of normal or healthy weight, overweight, obese or extremely obese. In an embodiment, the adult receiving the treatment has a BMI classification of normal with a BMI between 18.5 kg/m² and 24.9 kg/m². In an embodiment, the adult receiving the treatment has a BMI classification of overweight with a BMI≥25.0 kg/m². In an embodiment, the adult receiving the treatment has a BMI classification of pre-obese with a BMI between 25.0 kg/m² and 29.9 kg/m². In an embodiment, the adult receiving the treatment has a BMI classification as obese with a BMI≥30.0 kg/m².

In one aspect, a method for eradicating Helicobacter pylori infection in an obese adult preparing for bariatric surgery comprises confirming that the obese adult, having a body mass index ≥30 kg/m², is infected with Helicobacter pylori; requesting that the obese adult orally administer, for 14 days, a plurality of fixed-dose combination formulations which provide a total daily dose of 150 mg rifabutin, 3000 mg amoxicillin, and 120 mg omeprazole or an equivalent amount of a pharmaceutically acceptable salt thereof; and confirming that the Helicobacter pylori infection has been eradicated. In an embodiment, the administering comprises taking four fixed-dose combination formulations every 8 hours, wherein each dose of four fixed-dose combination formulations includes 50 mg rifabutin, 1,000 mg amoxicillin and 40 mg omeprazole or an equivalent amount of a pharmaceutically acceptable salt thereof. In an embodiment, each fixed- dose combination formulation comprises 12.5 mg rifabutin, 250 mg amoxicillin and 10 mg omeprazole or an equivalent amount of a pharmaceutically acceptable salt thereof. In an embodiment, each fixed-dose combination formulation is a capsule. In an embodiment, each fixed-dose combination formulation comprises a mixture of amoxicillin/rifabutin immediate release minitablets and enteric-coated omeprazole minitablets. In an embodiment, each minitablet is about 2 mm in size. In an embodiment, each fixed-dose combination formulation comprises between 36 and 40 amoxicillin/rifabutin immediate release minitablets and between 32 and 36 enteric-coated omeprazole minitablets. In an embodiment, each fixed-dose combination formulation comprises 38 amoxicillin/rifabutin immediate release minitablets and 34 enteric-coated omeprazole minitablets. In an embodiment, at least 70% by weight of the omeprazole is intestinally released after oral administration. In an embodiment, the amoxicillin is amoxicillin trihydrate. In an embodiment, the omeprazole is omeprazole magnesium. In an embodiment, the confirming that the obese adult is infected with Helicobacter pylori is carried out with a 13 C UBT or fecal antigen test. In an embodiment, the confirming that the Helicobacter pylori infection has been eradicated is carried out with a ¹³C UBT or fecal antigen test performed ≥28 days post-therapy.

In one aspect, a method for preparing an obese adult for bariatric surgery comprises testing the obese adult having a body mass index ≥30 kg/m² for infection with Helicobacter pylori, and if the obese adult is confirmed to have Helicobacter pylori, providing the obese adult with a plurality of fixed-dose combination formulations which provide a total daily dose of 150 mg rifabutin, 3000 mg amoxicillin, and 120 mg omeprazole or an equivalent amount of a pharmaceutically acceptable salt thereof for a total of 14 days; and confirming that the Helicobacter pylori infection has been eradicated. In an embodiment, four fixed-dose combination formulations are administered to the obese adult every 8 hours, wherein each dose of four fixed- dose combination formulations includes 50 mg rifabutin, 1,000 mg amoxicillin and 40 mg omeprazole or an equivalent amount of a pharmaceutically acceptable salt thereof. In an embodiment, each fixed-dose combination formulation comprises 12.5 mg rifabutin, 250 mg amoxicillin and 10 mg omeprazole or an equivalent amount of a pharmaceutically acceptable salt thereof. In an embodiment, each fixed-dose combination formulation is a capsule. In an embodiment, each fixed-dose combination formulation comprises a mixture of amoxicillin/rifabutin immediate release minitablets and enteric-coated omeprazole minitablets. In an embodiment, each minitablet is about 2 mm in size. In an embodiment, each fixed-dose combination formulation comprises between 36 and 40 amoxicillin/rifabutin immediate release minitablets and between 32 and 36 enteric-coated omeprazole minitablets. In an embodiment, each fixed-dose combination formulation comprises 38 amoxicillin/rifabutin immediate release minitablets and 34 enteric-coated omeprazole minitablets. In an embodiment, at least 70% by weight of the omeprazole is intestinally released after oral administration. In an embodiment, the amoxicillin is amoxicillin trihydrate. In an embodiment, the omeprazole is omeprazole magnesium. In an embodiment, the confirming that the obese adult is infected with Helicobacter pylori is carried out with a ¹³C UBT or fecal antigen test. In an embodiment, the confirming that the Helicobacter pylori infection has been eradicated is carried out with a ¹³C UBT or fecal antigen test performed ≥28 days post-therapy.

In one aspect, a method of eradicating Helicobacter pylori infection in an obese adult during the pre-operative phase of a bariatric surgery intervention comprises testing the obese adult having a body mass index ≥30 kg/m 2 for infection with Helicobacter pylori, and if the obese adult is confirmed to have Helicobacter pylori, providing the obese adult with a plurality of fixed-dose combination formulations which provide a total daily dose of 150 mg rifabutin, 3000 mg amoxicillin, and 120 mg omeprazole or an equivalent amount of a pharmaceutically acceptable salt thereof for a total of 14 days; and confirming that the Helicobacter pylori infection has been eradicated. In an embodiment, four fixed-dose combination formulations are administered to the obese adult every 8 hours, wherein each dose of four fixed-dose combination formulations includes 50 mg rifabutin, 1,000 mg amoxicillin and 40 mg omeprazole or an equivalent amount of a pharmaceutically acceptable salt thereof. In an embodiment, each fixed-dose combination formulation comprises 12.5 mg rifabutin, 250 mg amoxicillin and 10 mg omeprazole or an equivalent amount of a pharmaceutically acceptable salt thereof. In an embodiment, each fixed- dose combination formulation is a capsule. In an embodiment, each fixed-dose combination formulation comprises a mixture of amoxicillin/rifabutin immediate release minitablets and enteric-coated omeprazole minitablets. In an embodiment, each minitablet is about 2 mm in size. In an embodiment, each fixed-dose combination formulation comprises between 36 and 40 amoxicillin/rifabutin immediate release minitablets and between 32 and 36 enteric-coated omeprazole minitablets. In an embodiment, each fixed-dose combination formulation comprises 38 amoxicillin/rifabutin immediate release minitablets and 34 enteric-coated omeprazole minitablets. In an embodiment, at least 70% by weight of the omeprazole is intestinally released after oral administration. In an embodiment, the amoxicillin is amoxicillin trihydrate. In an embodiment, the omeprazole is omeprazole magnesium. In an embodiment, the confirming that the obese adult is infected with Helicobacter pylori is carried out with a ¹³C UBT or fecal antigen test. In an embodiment, the confirming that the Helicobacter pylori infection has been eradicated is carried out with a ¹³C UBT or fecal antigen test performed ≥28 days post-therapy.

A fixed-dose combination formulation comprising 12.5 mg rifabutin, 250 mg amoxicillin and 10 mg omeprazole or an equivalent amount of a pharmaceutically acceptable salt thereof for use in the treatment of H. pylori infection in an obese patient, wherein the obese patient has a body mass index ≥30 kg/m².

A fixed-dose combination formulation comprising 12.5 mg rifabutin, 250 mg amoxicillin and 10 mg omeprazole or an equivalent amount of a pharmaceutically acceptable salt thereof for use in the treatment of H. pylori infection in an obese patient preparing for bariatric surgery, wherein the obese patient has a body mass index ≥30 kg/m².

A fixed-dose combination formulation comprising 12.5 mg rifabutin, 250 mg amoxicillin and 10 mg omeprazole or an equivalent amount of a pharmaceutically acceptable salt thereof for use as a medicament in the treatment of H. pylori infection in an obese patient having a body mass index ≥30 kg/m².

A fixed-dose combination formulation comprising 12.5 mg rifabutin, 250 mg amoxicillin and 10 mg omeprazole or an equivalent amount of a pharmaceutically acceptable salt thereof for use as a medicament in the treatment of H. pylori infection in an obese patient preparing for bariatric surgery, wherein the obese patient has a body mass index ≥30 kg/m².

A fixed-dose combination formulation comprising 12.5 mg rifabutin, 250 mg amoxicillin and 10 mg omeprazole or an equivalent amount of a pharmaceutically acceptable salt thereof in preparation of a medicament in the treatment of H. pylori infection in an obese patient having a body mass index ≥30 kg/m².

A fixed-dose combination formulation comprising 12.5 mg rifabutin, 250 mg amoxicillin and 10 mg omeprazole or an equivalent amount of a pharmaceutically acceptable salt thereof in preparation of a medicament in the treatment of H. pylori infection in an obese patient preparing for bariatric surgery, wherein the obese patient has a body mass index ≥30 kg/m².

### BRIEF DESCRIPTION OF THE DRAWINGS

The presently disclosed embodiments will be further explained with reference to the drawings.

FIG. 1 shows a Forest Plot in the intent-to-treat (ITT) population for two studies that compared the success (%) of H. pylori eradication by three (3) body mass index subgroups. Solid circles represent hazard ratios (95% CI).

FIG. 2 shows a Forest Plot in the intent-to-treat (ITT) population for two studies that compared the success (%) of H. pylori eradication by two (2) body mass index subgroups. Solid circles represent hazard ratios (95% CI).

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION OF THE INVENTION

Obesity is most often classified using body mass index (BMI) (WHO classification):

| BMI, kg/m² | WHO classification |
|---|---|
| Below 18.5 | Underweight |
| 18.50-24.99 | Normal or Healthy Weight |
| 25.0-29.99 | Overweight |
| 30.00-34.99 | Obese class I |
| 35.00-39.99 | Obese class II |
| 40.00 or greater | Obese class III (morbidly |
| | obese, extremely obese) |

As used herein, empiric triple H. pylori therapy consists of a proton pump inhibitor (PPI) plus two antibiotics (amoxicillin with clarithromycin or metronidazole)

As used herein, empiric concomitant quadruple H. pylori therapy consists of a PPI plus three antibiotics (amoxicillin, clarithromycin, and metronidazole.

As used herein, a “fixed-dose combination” is two or more drugs contained in a single dosage form, such as a capsule or tablet.

As used herein, “did not work as well”, means that the same exact therapy (e.g., same drugs, same dosage strengths of drugs, same dosing schedule of drugs, and same amount of treatment in days/weeks) was inferior by at least 15% in a patient having a BMI in the overweight/obese category as compared to a patient having a BMI in the normal category. In an embodiment, “did not work as well”, means that the same exact therapy (e.g., same drugs, same dosage strengths of drugs, same dosing schedule of drugs, and same amount of treatment in days/weeks) was inferior by at least 20% in a patient having a BMI in the overweight/obese category as compared to a patient having a BMI in the normal category. In an embodiment, “did not work as well”, means that the same exact therapy (e.g., same drugs, same dosage strengths of drugs, same dosing schedule of drugs, and same amount of treatment in days/weeks) was inferior by at least 25% in a patient having a BMI in the overweight/obese category as compared to a patient having a BMI in the normal category.

As used herein, “substantially equivalent”, means that the eradication rate of a patient within any BMI classification as compared to another BMI classification is within 10% on the same exact therapy (e.g., same drugs, same dosage strengths of drugs, same dosing schedule of drugs, and same amount of treatment in days/weeks) and thus is not substantially different.

As used herein, the intent-to-treat (ITT) population (also known as full analysis set (FAS) population) includes all subjects who took at least one dose of study drug.

As used herein, the modified intent-to-treat (mITT) population includes all subjects who took at least one dose of study drug and who returned for follow up test of cure.

Rifabutin-based triple therapy for treating H. pylori infection in adults is marketed in the U.S. by RedHill Biopharma Inc. and under the brand name Talicia®. Talicia® is a fixed-dose combination (FDC) capsule comprising omeprazole magnesium, amoxicillin and rifabutin for oral administration and is described, for example, in U.S. Pat. No. 9,050,263; U.S. Pat. No. 9,498,445; U.S. Pat. No. 9,603,806; and U.S. Pat. No. 10,238,606, all of which are specifically incorporated by reference.

Talicia® delayed-release capsules contain a mixture of amoxicillin/rifabutin immediate release minitablets and enteric-coated omeprazole minitablets. Each minitablet is a compressed tablet with a typical diameter of one to four millimeters, and more preferably about 2.45 mm±0.15 mm. Early clinical batches were performed on a mini-encapsulator, which was semi-automatic. The capsules were filled by allowing an equal weight of each type of minitablet to be encapsulated, but the number of each type of minitablet was not controlled. To provide additional control over the actual number of each type of minitablet encapsulated, rather than just equivalent weights, the equipment selected for use in the later clinical and production batches employs a dosing wheel to ensure a maximum of 38 rifabutin/amoxicillin minitablets, and 34 delayed-release omeprazole minitablets, are encapsulated. The Talicia® delayed-release capsules are filled using an encapsulator fitted with two minitablet hoppers and a microtablet dosing unit. The unit is comprised of a wheel with a predefined number of holes, the dimensions of which are dictated by the size of the minitablets. The minitablets enter into the wheel holes by means of a vacuum and a brush eliminates excess minitablets. The wheel rotates, the vacuum is cut off and the minitablets fall down into the drum. One row of rifabutin/amoxicillin minitablets are released into each gelatin capsule (along with the contents of the second wheel, containing the delayed-release omeprazole minitablets). During the filling process, some holes could be empty or minitablets might not be released into the capsule if the tablet becomes stuck. As a result, filled capsules have a maximum of 38 amoxicillin/rifabutin minitablets and 34 omeprazole minitablets, but may actually have less. The target weight for each Talicia® capsule is calculated on a per batch basis, taking into account the average weight of the minitablets used. In an embodiment, each FDC of the present invention comprises between 36 and 40 amoxicillin/rifabutin immediate release minitablets and between 32 and 36 enteric-coated omeprazole minitablets, and more preferably 38 amoxicillin/rifabutin immediate release minitablets and 34 enteric-coated omeprazole minitablets.

Omeprazole magnesium, a proton pump inhibitor, is included in the delayed-release component of the capsule, and amoxicillin and rifabutin, antibacterial drugs, are included in the immediate-release component of the capsule. Each delayed-release capsule, which is a fixed-dose combination formulation, contains:
omeprazole 10 mg (equivalent to 10.3 mg of omeprazole magnesium)
amoxicillin 250 mg (equivalent to 286.9 mg of amoxicillin trihydrate)
rifabutin 12.5 mg

Each Talicia® delayed-release capsule further contains the following inactive ingredients: crospovidone, FD&C Red 3, FD&C Yellow 6, gelatin, hydroxypropyl cellulose, hypromellose, magnesium stearate, mannitol-starch, methacrylic acid copolymer, meglumine, pregelatinized starch, silica, sodium bicarbonate, sodium lauryl sulfate, talc, titanium dioxide and triethyl citrate.

According to its approved use, four (4) Talicia® capsules should be administered every 8 hours for 14 days with food and a full glass of water (8 ounces). Each dose (4 capsules) of Talicia® includes rifabutin 50 mg, amoxicillin 1,000 mg and omeprazole 40 mg.

### Omeprazole Magnesium

Omeprazole magnesium is a white to off-white powder with a melting point with degradation at 200° C. The salt is slightly soluble (0.25 mg/mL) in water at 25 ° C., and it is soluble in methanol. Omeprazole magnesium is 5-methoxy-2-[[(4-methoxy-3,5-dimethyl-2-pyridyl) methyl] sulfinyl]benzimidazole, (RS) magnesium salt (2:1). Omeprazole magnesium has a molecular formula of (C₁₇H₁₉N₃O₃S)₂ Mg, and a molecular weight of 713.12. The structural formula is:

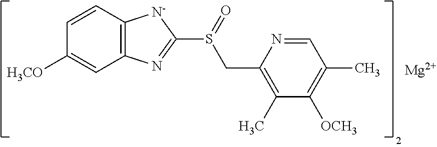

### Amoxicillin

Amoxicillin is a semisynthetic antibacterial drug, an analog of ampicillin Chemically it is (2S,5R,6R)-6-RR)-(-)-2- amino-2-(p-hydroxyphenyl)acetamido1-3,3-dimethyl-7-oxo-4-thia-1- azabicyclo[3.2.0] heptane-2-carboxylic acid trihydrate. Amoxicillin has a molecular formula of C₁₆H₁₉N₃O₅S·3 H₂O, and a molecular weight of 419.45. The structural formula is:

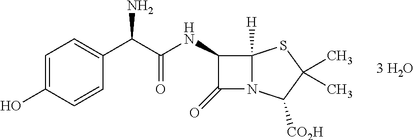 [SMILES: CC1(C)S[C@@H]2[C@H](NC(=O)[C@H](N)c3ccc(O)cc3)C(=O)N2[C@H]1C(=O)O]

Amoxicillin is a broad antimicrobial beta-lactam that inhibits the synthesis of the bacterial cell wall in replicating bacteria. Amoxicillin is bactericidal for both gram-positive and gram-negative bacteria and is destroyed by beta-lactamase produced from both types of bacteria.

### Rifabutin

Rifabutin is a red-violet powder soluble in chloroform and methanol, sparingly soluble in ethanol, and very slightly soluble in water (0.19 mg/mL). Its log P value (the base 10 logarithm of the partition coefficient between n-octanol and water) is 3.2 (n-octanol/water).

Rifabutin is (9S,12E,14S,15R,16S,17R,18R,19R,20S,21S,22E,24Z)-6-16,18,20-tetrahydroxy-l′-isobutyl-14-methoxy-7,9,15,17,19,21,25- heptamethylspiro [9,4-(epoxypentadeca[1,11,13] trienimino) -2H-furo [2′,3′ :7,8] naphth [1,2-d] imidazole-2,4′-piperidine]-5,10,26-(3H,9H)-trione-16-acetate. Rifabutin has a molecular formula of C₄₆H₆₂N₄O₁₁, and a molecular weight of 847.02. The structural formula is:

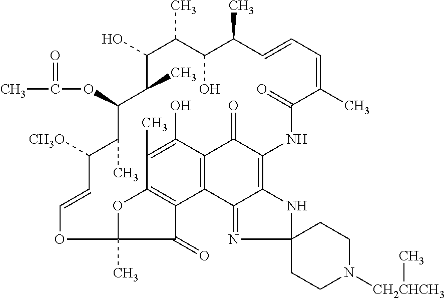

The effectiveness and safety of Talicia® were evaluated in a randomized, double-blind, controlled study of Talicia® in treatment-naïve H. pylori-positive adult patients complaining of epigastric pain/discomfort (Study 1). H. pylori infection at baseline was defined as positive by ¹³C urea breath test (UBT) and follow-up upper endoscopy (culture, histology, or Campylobacter-like organism test). Patients were randomized 1:1 to Talicia® or control (total daily dose of amoxicillin 3000 mg and omeprazole 120 mg) administered for 14 consecutive days. The trial was performed in the U.S and designed to evaluate the added contribution of rifabutin to the Talicia® triple combination.

H. pylori eradication was confirmed with a negative ¹³C UBT or fecal antigen test performed ≥28 days post-therapy and suspended PPI. Patients with negative test results were considered treatment successes. Patients who tested positive for H. pylori infection were considered treatment failures, and patients with indeterminate, not assessable, or missing results from the test of cure visits underwent a repeat ¹³ C UBT test. Persistent indeterminate results and patients without any ¹³C UBT or fecal antigen test after baseline were considered as treatment failures.

H. pylori eradication rates are shown in Table 1. The difference in response rates between

Talicia® and the control was 26.1% (95% CI; 18.0, 34.1).

TABLE 1
Eradication Rates of H. pylori in Study 1

| | ITT Population^{a} | |
|---|---|---|
| H. pylori | Talicia ® | Control |
| Eradication | N = 228 (%) | N = 227 (%) |
| Success | 191 (83.8) | 131 (57.7) |
| Failure | 37 (16.2)^{b} | 96 (42.3) |
| P-value | <0.0001 | |

^{a}The Intent to Treat (ITT) population included all randomized patients who received at least one dose of study drug.
^{b}Of those subjects classified as treatment failures, all but one subject in the Talicia ® group were positive by ¹³C UBT; this one subject was classified as a treatment failure due to a missing post-baseline test result.

A randomized, double-blind, placebo-controlled study of Talicia® in H. pylori-positive adult patients complaining of epigastric pain/discomfort (Study 2) was performed in the U.S. and provided supportive evidence for the efficacy of Talicia® for the treatment of H. pylori infection; 77 patients taking Talicia® and 41 patients taking placebo were included in the ITT population, with an eradication rate of 76.6% (95% CI; 66.0%, 84.7%) for the Talicia® -treated patients compared to 2.4% for the placebo-treated patients. Eleven patients in the Talicia® arm and four patients in the placebo arm were classified as treatment failures due to missing ¹³C UBT results at the test-of-cure visit.

In the past, medical professionals have described the need for a patient-tailored H. pylori eradication protocol for obese patients about to undergo bariatric surgery to improve the eradication rate, which is reportedly insufficient compared with that in the non-obese population. Abdullahi et al. conducted a pilot study to evaluate whether body mass index (BMI) might influence the success rate of H. pylori eradication (OBES SURG (2008) 18: 1450-1454). In that pilot study, eighty-one nondiabetic naïve H. pylori-positive patients were divided into two groups according to their BMI, with 41 in the control group (normal BMI) and 40 in the overweight/obese group (BMI, of ≥25). Both groups were given a triple therapy consisting of pantoprazole 40 mg, as proton pump inhibitor, for 2 weeks plus amoxicillin 1 g t.i.d, and clarithromycin 250 mg t.i.d, as antibacterial agents, for the first week of treatment. Eradication was confirmed by the ¹³C-urea breath test at 3 months. In that pilot study, successful eradication was observed in 55.0% of the overweight/obese group (Body Mass Index, BMI, of ≥25) compared with 85.4% in the normal BMI group. [p<0.005; odds ratio (OR): 4.77; 95% confidence interval (CI): 1.64-13.87].

FDA Phase III studies may or may not establish that a drug works to treat a particular disease or condition. Moreover, prior to completion and evaluation of Phase III, a person of skill in the art would not necessarily understand that the drug is useful for treatment of the disease or condition or that the drug can be administered “as-is” to any patient regardless of BMI.

Data was pooled from the two above-mentioned Phase 3 clinical studies (Study 1 and Study 2) assessing H. pylori eradication with RHB-105 (an ‘all-in-one’ low-dose rifabutin therapy providing 50 mg rifabutin, 1,000 mg amoxicillin, and 40 mg omeprazole) vs an active comparator of a high dose PPI-amoxicillin (1,000 mg amoxicillin and 40 mg omeprazole) q8h for 14 days. Treatment naïve subjects with confirmed H. pylori infection were treated and eradication verified using ¹³C UBT (urea breath test) at least 4 weeks post-therapy as a test-of-cure. An analysis was performed on the pooled Intent-to-Treat (ITT) population and calculated eradication rates based upon BMI cut points: <30 kg/m 2 representing overweight and below, obese ≥30 kg/m 2 and <40 kg/m², and extremely obese ≥40 kg/m² BMI measures (Table 2) and BMI cut points: <40 kg/m ² (normal, overweight and obese) and extremely obese ≥40 kg/m² BMI measures (Table 3). A comparison was made between the RHB-105 and the active comparator treatment groups using pooled ITT and other analyses.

In the pooled analysis, there were 532 evaluable subjects (305 pooled RHB-105 treatment group and 227 in the active comparator group). The mean age was 45.9 vs. 47.2 years. Other characteristics were generally similar with and a mean BMI of 30.4 kg/m² vs. 30.9 kg/m². Looking more closely at eradication rates based upon 3 BMI subgroups (Table 2), pooled RHB-105 rates were 82.4%, 84.6%, 90.9% for BMI <30, ≥30 and <40, ≥40 vs 58.6%, 62.9%, 31.8% (Active Comparator), respectively. Looking more closely at eradication rates based upon 2 BMI subgroups (Table 3), pooled RHB-105 rates were 81.3% and 90.9% for BMI <40, ≥40 vs 60.5% and 31.8% (Active Comparator), respectively. RHB-105 eradication was statistically significant for all BMI subgroups over the active comparator (P<0.0001). As such, BMI does not appear to influence eradication rates among the RHB-105 group while there was a ˜50%-60% drop in the active comparator eradication for the highest BMI group.

TABLE 2
BMI Influence on H. pylori Eradication Success Rate (ITT) - 3 BMI Subgroups

| | | | | Study RHB-105-02 |
|---|---|---|---|---|
| | | Study RHB-105-02 | Pooled | Active |
| | | RHB-105 | RHB-105 | Comparator |
| BMI Group | Assessment | (N = 228) | (N = 305) | (N = 227) |
| BMI <= 30 | n | 118 | 153 | 116 |
| | Treatment Success | 95 (80.5%) | 126 (82.4%) | 68 (58.6%) |
| | 95% CI | 72.4-86.6 | 75.5-87.6 | 49.5-67.2 |
| 30 < BMI < 40 | n | 94 | 123 | 89 |
| | Treatment Success | 82 (87.2%) | 104 (84.6%) | 56 (62.9%) |
| | 95% CI | 79.0-92.5 | 77.1-89.9 | 52.5-72.2 |
| BMI >= 40 | n | 16 | 22 | 22 |
| | Treatment Success | 14 (87.5%) | 20 (90.9%) | 7 (31.8%) |
| | 95% CI | 64.0-96.5 | 72.2-97.5 | 16.4-52.7 |

TABLE 3
BMI Influence on H. pylori Eradication Success Rate (ITT) - 2 BMI Subgroups

| | | | | Study RHB-105-02 |
|---|---|---|---|---|
| | | Study RHB-105-02 | Pooled | Active |
| | | RHB-105 | RHB-105 | Comparator |
| BMI Group | Assessment | (N = 228) | (N = 305) | (N = 227) |
| BMI < 40 | n | 212 | 283 | 205 |
| | Treatment Success | 177 (83.5%) | 230 (81.3%) | 124 (60.5%) |
| | 95% CI | 77.9-87.9 | 76.3-85.4 | 53.7-66.9 |
| 40 <= BMI | n | 16 | 22 | 22 |
| | Treatment Success | 14 (87.5%) | 20 (90.9%) | 7 (31.8%) |
| | 95% CI | 64.0-96.5 | 72.2-97.5 | 16.4-52.7 |

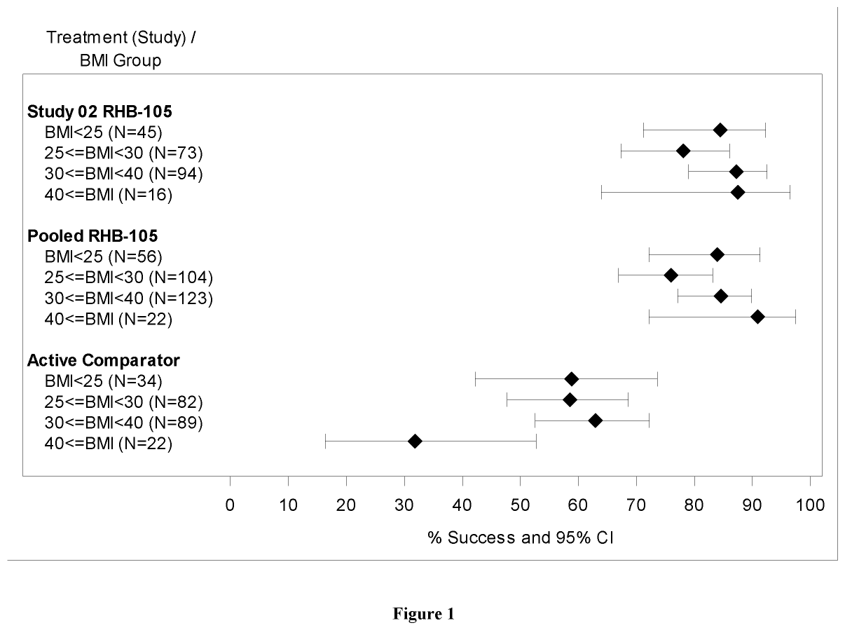

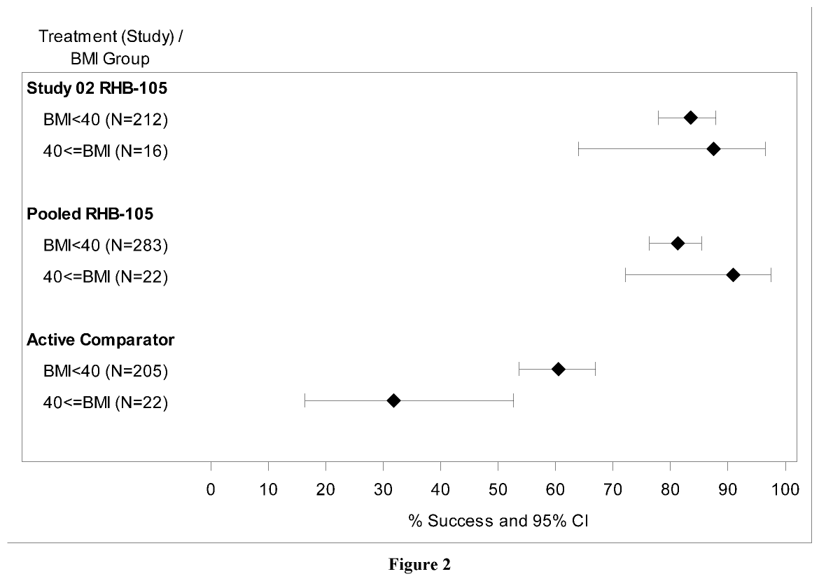

FIG. 1 shows a Forest Plot in the intent-to-treat (ITT) population for two studies that compared the success (%) of H. pylori eradication by three (3) body mass index subgroups. Solid circles represent hazard ratios (95% CI). FIG. 2 shows a Forest Plot in the intent-to-treat (ITT) population for two studies that compared the success (%) of H. pylori eradication by two (2) body mass index subgroups. Solid circles represent hazard ratios (95% CI).

Eradication rates of the Active Comparator were greatly affected by increase in BMI. Rifabutin mitigated the BMI influenced failure of eradication seen with the active comparator for extremely obese subjects. The addition of low-dose rifabutin demonstrated a statistically significant improvement and clinically meaningful eradication vs the active comparator. RHB -105 may serve as a first line empiric treatment of H. pylori infection and may be useful in eradication of H. pylori infection regardless of BMI status.

What is claimed is:

1. A method of treating patients who are positive for Helicobacter pylori infection comprising:
determining that a first patient who is positive for Helicobacter pylori infection has a body mass index corresponding to normal weight or overweight;
determining that a second patient who is positive for Helicobacter pylori infection has a body mass index corresponding to obese or extremely obese; and
administering to both the first patient and the second patient, for 14 days, a rifabutin-based triple therapy consisting essentially of rifabutin, amoxicillin and omeprazole or an equivalent amount of a pharmaceutically acceptable salt thereof, wherein, after treatment is complete, there is no substantial difference in the efficacy of the treatment when the treatment is administered to the first patient or the second patient.

2. The method of claim 1, wherein the first patient has a BMI ranging from 18.50 kg/m² to 24.99 kg/m² and the second patient has a BMI ranging from 30.0 kg/m² to 34.99 kg/m².

3. The method of claim 1, wherein the first patient has a BMI ranging from 18.50 kg/m² to 24.99 kg/m² and the second patient has a BMI ranging from 35.0 kg/m² to 39.99 kg/m².

4. The method of claim 1, wherein the first patient has a BMI ranging from 18.50 kg/m² to 24.99 kg/m² and the second patient has a BMI above 40.00 kg/m 2 .

5. The method of claim 1, wherein the first patient has a BMI ranging from 25.00 kg/m² to 29.99 kg/m² and the second patient has a BMI ranging from 35.0 kg/m² to 39.99 kg/m².

6. The method of claim 1, wherein the first patient has a BMI 25.00 kg/m² to 29.99 kg/m² and the second patient has a BMI ranging from 30.0 kg/m² to 34.99 kg/m².

7. The method of claim 1, wherein the first patient has a BMI 25.00 kg/m² to 29.99 kg/m² and the second patient has a BMI above 40.00 kg/m^{2 .}

8. The method of claim 1, wherein the rifabutin-based triple therapy comprises a plurality of fixed-dose combination formulations which provide a total daily dose of 150 mg rifabutin, 3,000 mg amoxicillin, and 120 mg omeprazole or an equivalent amount of a pharmaceutically acceptable salt thereof.

9. The method of claim 1, wherein the administering comprises taking four fixed-dose combination formulations every 8 hours, wherein each dose of four fixed-dose combination formulations includes 50 mg rifabutin, 1,000 mg amoxicillin and 40 mg omeprazole or an equivalent amount of a pharmaceutically acceptable salt thereof.

10. The method of claim 8, wherein each fixed-dose combination formulation comprises 12.5 mg rifabutin, 250 mg amoxicillin and 10 mg omeprazole or an equivalent amount of a pharmaceutically acceptable salt thereof.

11. The method of claim 8, wherein each fixed-dose combination formulation is a capsule.

12. The method of claim 8, wherein each fixed-dose combination formulation comprises a mixture of amoxicillin/rifabutin immediate release minitablets and enteric-coated omeprazole minitablets.

13. The method of claim 12, wherein each minitablet is about 2 mm in size.

14. The method of claim 8, wherein each fixed-dose combination formulation comprises between 36 and 40 amoxicillin/rifabutin immediate release minitablets and between 32 and 36 enteric- coated omeprazole minitablets.

15. The method of claim 8, wherein each fixed-dose combination formulation comprises 38 amoxicillin/rifabutin immediate release minitablets and 34 enteric-coated omeprazole minitablets.

16. The method of claim 8, wherein at least 70% by weight of the omeprazole is intestinally released after oral administration.

17. The method of claim 1, wherein the amoxicillin is amoxicillin trihydrate.

18. The method of claim 1, wherein the omeprazole is omeprazole magnesium.

19. The method of claim 1, further comprising confirming H. pylori eradication with a negative ¹³C UBT or fecal antigen test performed ≥28 days post-therapy.

20. The method of claim 1, wherein the second patient who tested positive for Helicobacter pylori infection is obese with a BMI above 30.00 kg/m² and is preparing for bariatric surgery.
